(19) United States
(12) Patent Application Publication
(10) Pub. No.: US 2007/0176131 A1
(43) Pub. Date: Aug. 2, 2007

(54) Radiation image information detecting panel and radiation image information read-out system

(75) Inventor: Kenji Takahashi, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation

(21) Appl. No.: 11/591,599

(22) Filed: Nov. 2, 2006

(30) Foreign Application Priority Data
Nov. 2, 2005 (JP) 319002/2005

(51) Int. Cl. G21K 4/00; G01N 23/04; G03C 5/16; G02B 26/10; A61B 6/00

(52) U.S. Cl. 250/581; 250/591

(57) ABSTRACT

A radiation absorbing phosphor layer including phosphors which absorb radiation and emit light according to the amount of the radiation, a stimulable photoconductive layer which generates electron-positive hole charges in an amount according to the amount of light and stores and records a radiation image information by trapping the charges while releasing the trapped charges upon exposure to stimulating light, a pair of photocurrent detecting electrode layers which are permeable to light and are respectively provided on upper and lower surfaces of the stimulable photoconductive layer to detect the released charges, and a panel-like light source for projecting the stimulating light onto the stimulable photoconductive layer is provided to form a radiation image information detecting panel.

### BACKGROUND OF THE INVENTION

1. Field of the Invention

This invention relates to a radiation image information detecting panel for storing and recording a radiation image, and a radiation image information read-out system provided with the panel.

2. Description of the Related Art

There has been known as an image recording medium for recording a radiation image, for instance, in taking a medical x-ray image, a stimulable phosphor sheet which stores and records (the primary stimulation) the radiation energy according to a radiation image and emits stimulated light according to the stored radiation energy when it is scanned by secondary stimulating light and exposed thereto (the secondary stimulation), and/or an electrostatic latent image recording sheet which records an image information as an electrostatic latent image and generates an electric current according to the electrostatic latent image when it is scanned by reading light and exposed thereto.

When an image information is to be read out from such an image recording medium, the reading light or the secondary stimulating light is generally scanned along the image recording medium to expose it.

When reading out an image from an image information detecting sheet (an electrostatic recording sheet) of a type disclosed in U.S. Pat. No. 6,376,857 where a radiation image is recorded as an electrostatic latent image, an electric current generated in the electrostatic recording sheet in response to projection of a reading light onto the electrostatic recording sheet is directly detected. In U.S. Pat. No. 6,376,857, there is proposed an image read-out system where a panel-like light source provided with an electroluminescence layer (EL layer) as a light source for emitting the reading light is integrated with an image recording medium of a type where an electrostatic latent image is recorded. In the case of an electrostatic recording sheet, since it is necessary to apply an electric field to the sheet in synchronization with the photographing unit (the radiation source) when photographing (when a latent image is recorded) and the sheet must be connected to the power source upon photography, a labor for wiring and the like is required upon photography.

In Japanese Unexamined Patent Publication No. 2000-338297, there is disclosed an image information detecting sheet (stimulable phosphor sheet) of a type where radiation energy is stored, and radiation image information detecting panel where a photoconductive layer which has substantially the same area with the stimulable phosphor sheet and has a sensitivity to the stimulated light is integrated with the stimulable phosphor sheet. However, since an image is read out from the sheet by reading the stimulated light generated in response to projection of the secondary stimulating light, the stimulated light and the secondary stimulating light mingle with each other when reading out the image. Accordingly, in order to accurately (with an excellent S/N) read out an image in the radiation image information detecting panel having a photoconductive layer and a stimulable phosphor layer integrated with each other, it is necessary to finely divide the stimulated light and the secondary stimulating light to suppress the influence of the secondary stimulating light and to detect only the stimulated light.

Whereas, in U.S. Pat. No. 7,087,915 there is disclosed a radiation image information detecting panel where a stimulable phosphor layer which exhibits a Gudden-Pohl effect and emits stored energy as light in response to stimulation by an electric field is provided, and light generated in response to stimulation by an electric field is detected by a two-dimensional detecting layer including a photoconductive layer. In accordance with this radiation image information detecting panel, since the stimulable phosphor layer is stimulated by applying an electric field instead of projecting the secondary stimulating light, the problem of mingling the secondary stimulating light with the stimulated light arises and it is not necessary to divide the secondary stimulating light and the stimulated light, which has been a problem in Japanese Unexamined Patent Publication No. 2000-338297.

In Japanese Unexamined Patent Publication No. 7(1995)-092023, there is disclosed an electronic device where a quantity of first light is measured by projecting the first light having photon energy larger than the energy band gap of material such as a semiconductor having a photoelectric effect and trap levels onto the material, subsequently projecting second light having photon energy smaller than the energy band gap of the material and measuring the photocurrent generated in response to projection of the second light, and proposed, for instance, use as an actinometer and a memory. This is on the basis of the principle that electron-hole electric charges are generated in the material in response to projection of the first light according to the quantity of the first light, and the electric charges are trapped by the trap levels and released from the trap levels in response to projection of the second light. The electric charges generated in response to projection of the second light can be detected as the photocurrent by applying an electric field and since the amount of the photocurrent depends upon the quantity of the first light, information on the first light can be obtained by projection of the second light.

Recently, a radiation image information detecting panel is required to be easy to handle, simple in structure and to able to be used as a cassette which is asynchronous with the photographing unit (the radiation source) and a radiation image information read-out system is required to be simple in structure where division of the stimulated light from the stimulating light is unnecessary and inexpensive.

### SUMMARY OF THE INVENTION

In view of the foregoing observations and description, the primary object of the present invention is to provide a radiation image information detecting panel which is easy to handle and simple in structure, and accordingly, high in productivity and inexpensive.

Another object of the present invention is to provide a radiation image information read-out system which employs the radiation image information detecting panel and is simple in structure and inexpensive.

In accordance with the present invention, there is provided a first radiation image information detecting panel comprising

a radiation absorbing phosphor layer including phosphors which absorb radiation and emit light according to the amount of the radiation,

a stimulable photoconductive layer which generates electron-positive hole charges in an amount according to the amount of light and stores and records a radiation image information by trapping the charges while releasing the trapped charges upon exposure to stimulating light,

a pair of photocurrent detecting electrode layers which are permeable to light and are respectively provided on upper and lower surfaces of the stimulable photoconductive layer to detect the released charges, and

a panel-like light source for projecting the stimulating light onto the stimulable photoconductive layer.

As the panel-like light source, those which comprise an electroluminescence layer emitting the stimulating light and a pair of electric field applying electrode layers at least the stimulable photoconductive layer side one of which is permeable to light and which are respectively provided on upper and lower surfaces of the electroluminescence layer to apply an electric field to the electroluminescence layer are preferred. Further, in this case, it is preferred that at least one of the photocurrent detecting electrode layers forms a first stripe electrode layer comprising a plurality of linear electrodes disposed parallel to each other and the other one of the photocurrent detecting electrode layers forms a second stripe electrode layer comprising a plurality of linear electrodes disposed parallel to each other, the linear electrodes of the first stripe electrode being in perpendicular to those of the second stripe electrode.

The one of the “pair of photocurrent detecting electrode layers which are permeable to light” disposed on the side thereof nearer to the radiation absorbing phosphor layer is permeable to, at least, the light emitted from the radiation absorbing phosphor layer and the other of the “pair of photocurrent detecting electrode layers which are permeable to light” disposed on the side thereof nearer to the panel-like light source is permeable to, at least, the stimulating light emitted from the panel-like light source. The “electric field applying electrode layers” are permeable to at least the light emitted from the electroluminescence layer.

In accordance with the present invention, there is further provided a first radiation image information read-out system comprising a first radiation image information detecting panel of the present invention and an image signal obtaining means which obtains an image signal carrying thereon image information by controlling the panel-like light source to cause the stimulating light to scan the stimulable photoconductive layer and detecting the charge released from the stimulable photoconductive layer in response to exposure to the stimulating light as a photocurrent by way of the photocurrent detecting electrode layers.

In accordance with the present invention, there is provided a second radiation image information detecting panel comprising

a radiation absorbing phosphor layer including phosphors which absorb radiation and emit light according to the amount of the radiation,

a stimulable photoconductive layer which generates electron-positive hole charges in an amount according to the amount of light and stores and records a radiation image information by trapping the charges while releasing the trapped charges upon application of an electric field,

a pair of photocurrent detecting electrode layers which are respectively provided on upper and lower surfaces of the stimulable photoconductive layer to apply the electric field and detect the charges, at least the stimulable photoconductive layer side one of the photocurrent detecting electrode layers being permeable to light.

It is preferred that the pair of photocurrent detecting electrode layers be of stripe electrodes each of which comprises a plurality of linear electrodes parallel to each other, and disposed so that the linear electrodes thereof extends perpendicularly to each other.

In accordance with the present invention, there is further provided a second radiation image information read-out system comprising a second radiation image information detecting panel of the present invention and an image signal obtaining means which obtains an image signal carrying thereon image information by controlling the electric field applied to the stimulable photoconductive layer and detecting the charge released from the stimulable photoconductive layer in response to application of the electric field by way of the photocurrent detecting electrode layers.

In the first radiation image information detecting panel of the present invention, there is provided stimulable photoconductive layer where radiation image information is stored and recorded by trapping the electric charges and the trapped electric charges are released upon exposure to the stimulating light. An image signal carrying thereon radiation image information can be obtained by detecting as a photocurrent the electric charges generated in response to scan of the stimulating light. Accordingly, the number of layers can be smaller than conventional panels which is provided with a stimulable phosphor layer and a photoconductive layer which exhibits a conductivity in response to light from the stimulable phosphor layer. Further, since it is unnecessary to provide a structure for separating the stimulated light from the stimulating light, it is simple in structure and high in productivity, whereby it is inexpensive. Further, since it is unnecessary to apply an electric field to the sheet when photographing, whereby the power source or the connection for this purpose need not be built in, the system can be compactly arranged, and since the panel can be used in asyncronization with the photographing unit, it is better to handle.

Since the first radiation image information read-out system of the present invention is provided with the first radiation image information detecting panel of the present invention and detects a photocurrent generated in the stimulable photoconductive layer in response to exposure to the stimulating light, deterioration of S/N due to mingle of the stimulated light with the stimulating light does not occur. Accordingly, S/N is better as compared with when the radiation image information is obtained by detecting the stimulating light, whereby an image signal good in S/N can be obtained.

In the second radiation image information detecting panel of the present invention, there is provided stimulable photoconductive layer which stores and records radiation image information by storing energy, and generates electric charges in response to application of an electric field, and an image signal carrying thereon radiation image information can be obtained by detecting as a photocurrent the electric charges generated in response to application of an electric field. Accordingly, a stimulating light source which has been necessary when the image information is obtained by stimulation of light becomes unnecessary and the number of layers can be smaller than conventional panels which is provided with a stimulable phosphor layer and a photoconductive layer which exhibits a conductivity in response to light from the stimulable phosphor layer. Further, since it is unnecessary to provide a structure for separating the stimulated light from the stimulating light, it is simple in structure and high in productivity, whereby it is inexpensive. Further, since it is unnecessary to apply an electric field to the sheet when photographing, whereby the power source or the connection for this purpose need not be built in, the system can be compactly arranged, and since the panel can be used in asyncronization with the photographing unit, it is better to handle.

Since the second radiation image information read-out system of the present invention is provided with the second radiation image information detecting panel of the present invention and detects a photocurrent generated in the stimulable photoconductive layer in response to application of an electric field, deterioration of S/N due to mingle of the stimulated light with the stimulating light does not occur. Accordingly, S/N is better as compared with when the radiation image information is obtained by detecting the stimulating light, whereby an image signal good in S/N can be obtained.

### BRIEF DESCRIPTION OF THE DRAWINGS

FIG. 1 is a perspective view of a radiation image information detecting panel in accordance with a first embodiment of the present invention,

FIG. 2 is a schematic view for illustrating a radiography by the use of the radiation image information detecting panel,

FIG. 3 is a view showing in brief a radiation image information read-out system employing the radiation image information detecting panel shown in FIG. 1,

FIG. 4 is a perspective view of a radiation image information detecting panel in accordance with a second embodiment of the present invention, and

FIG. 5 is a view showing in brief a radiation image information read-out system employing the radiation image information detecting panel shown in FIG. 4.

### DESCRIPTION OF THE PREFERRED EMBODIMENT

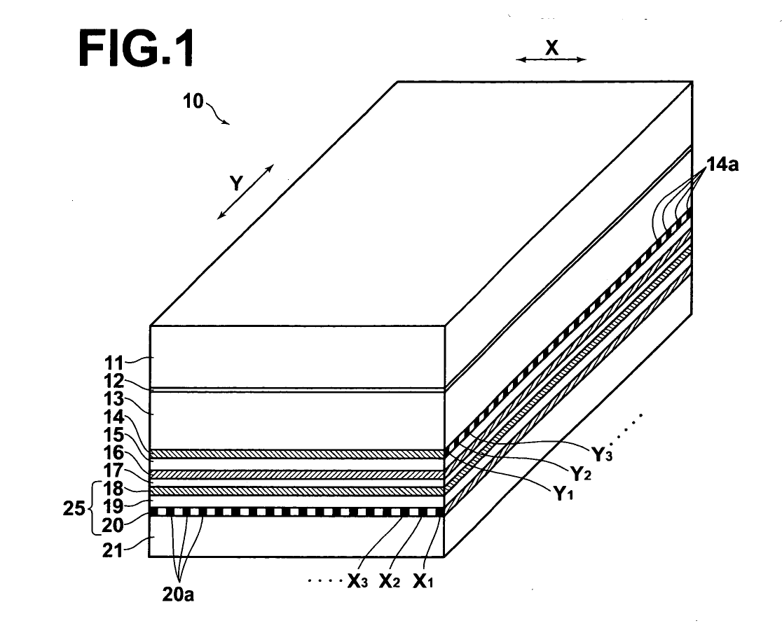

FIG. 1 is a perspective view showing a radiation image information detecting panel 10 in accordance with a first embodiment of the present invention. The radiation image information detecting panel 10 comprises a first support (base) 11, a reflective layer 12, a radiation absorbing phosphor layer 13, a first stripe electrode layer 14, a stimulable photoconductive layer 15, a first flat plate electrode layer 16, an insulating layer 17, a second flat plate electrode layer 18, an electroluminescence layer (EL layer) 19, a second stripe electrode layer 20, and a second support 21, which are superposed and integrated in this order. The radiation absorbing phosphor layer 13 includes therein phosphors which absorb radiation and emit light according to the amount of the radiation, the first stripe electrode layer 14 is formed by a number of linear electrodes (elements) 14a disposed like a stripe, the stimulable photoconductive layer 15 which absorbs light emitted from the radiation absorbing phosphor layer 13 to trap electron and/or positive holes while releases the trapped electron and/or positive holes upon exposure to stimulating light which is low in energy than the band gap, the EL layer 19 emits a secondary stimulating light to be projected onto the stimulable photoconductive layer 15, the second stripe electrode layer 20 is formed by a number of linear electrodes (elements) 20a disposed like a stripe, and the second support 21 is a protective layer. In this embodiment, a panel-like light source 25 is formed by the EL layer 19, and the second flat plate electrode layer 18 and the second stripe electrode layer 20 which are disposed upper and lower surfaces of the EL layer 19. In the panel-like light source 25, an insulating layer may be disposed each of between the EL layer 19 and upper electrode layer and between the EL layer 19 and lower electrode layer. Detail of each layer will be described later. In the panel 10 with this arrangement, it is preferred in obtaining a sharp image that the total thickness from the EL layer 19 to the stimulable photoconductive layer 15 is not larger than about 20 μm. When the flat plate electrode layers 16 and 18 are sufficiently thin with the insulating layer 17 and the EL layer 19 respectively set about 1 μm and 3 μm, the stimulable photoconductive layer 15 may be as thick as about 15 μm.

In the panel 10 of this embodiment, a pair of photocurrent detecting electrode layers for detecting electrons or the positive holes generated in the stimulable photoconductive layer 15 are formed by the first stripe electrode layer 14 and the first flat plate electrode layer 16 and a pair of electric field applying electrode layers for applying an electric field to the EL layer 19 are formed by the second stripe electrode layer 20 and the second flat plate electrode layer 18.

The elements 14a of the first stripe electrode layer 14 and the elements 20a of the second stripe electrode layer 20 cross with each other in perpendicular to the other as seen in plan. The elements 14a of the first stripe electrode layer 14 extends in the direction of arrow X from one end of the panel to the other at predetermined pitches in the direction of arrow Y (Y1, Y2, Y3, . . . ) and the elements 20a of the second stripe electrode layer 20 extends in the direction of arrow Y from one end of the panel to the other at predetermined pitches in the direction of arrow X (X1, X2, X3, . . . ). The pitches of the elements 20a of the second stripe electrode layer 20 and the pitches of the elements 14a of the first stripe electrode layer 14 govern the pixel pitches in the main scanning direction (the direction of arrow X) and the sub-scanning direction (the direction of arrow Y) and the elements are arranged in same number as the desired number of the pixels. For example, when linear electrodes (elements) 80 μm in width are arranged in parallel to each other at 100 μm pitches, the pixel pitches are then 100 μm. Of course, it is possible to cause a plurality of linear electrodes to correspond to one pixel.

The first stripe electrode 14 is permeable at least to light emitted from the radiation absorbing phosphor layer 11 and the first and second flat plate electrode layers 16 and 18 are permeable to light from the panel-like light source 25, that is, to light emitted from the EL layer. These electrodes permeable to light may be formed by an electrode partially exhibiting permeability to light such as a mesh-like or stripe-like metal electrode or a combination of these electrodes as well as ITO (Indium Tin Oxide), indium-doped zinc oxide film or metal film.

In the panel 10 of this embodiment, though an insulating layer 19 is provided between the first flat plate electrode layer 16 and the second flat plate electrode layer 18, one flat plate electrode layer may double the first flat plate electrode layer 16 and the second flat plate electrode layer 18 by providing no insulating layer. In this case, the single flat plate electrode layer should be sufficiently conductive.

Further, the first flat plate electrode layer may be a stripe electrode layer having a plurality of elements disposed in a similar manner opposed to a plurality of elements of the first stripe electrode layer. Further, the second flat plate electrode layer may be a stripe electrode layer having a plurality of elements disposed in a similar manner opposed to a plurality of elements of the second stripe electrode layer.

Further, it may possible for the first stripe electrode layer to be a flat plate electrode layer and for the second flat plate electrode layer to be a stripe electrode layer perpendicular to the second stripe electrode layer. In the case of such an electrode structure, the panel-like light source is equivalent to the light source where a number of point light sources are two-dimensionally arranged. However, it cannot be said to be more preferred since it is necessary for the amount light per one point to be very large in order to carry out a two-dimensional scanning of the point light source in a short time.

The first support 11 is of carbon and has a thickness of about 1 mm. For example, x-rays are projected as radiation carrying thereon image information from the side of this support 11.

The reflective layer 12 is a layer containing therein Al and SiO₂. The reflective layer 12 can be abbreviated. However, when a high-sensitive radiation sensor (e.g., a sensor for infants to minimize the exposing dose) is to be produced by the use of a low reflectance material such as a carbon plate as the first support 11, it is preferred that the reflective layer be provided. Whereas, in the case of high-sharpness type radiation sensor (e.g., a radiation sensor for mammography), the reflective layer is unnecessary.

As the radiation absorbing phosphor layer 13, for instance, those containing phosphors such as CsI:Na which absorbs x-rays and emits blue light and Lu₂O₃:Gd which absorbs x-rays and emits ultraviolet light may be employed. In addition to these phosphors, phosphors of LnTaO₄ series (including no impurities which functions as doping material, Ln representing a rare earth element), LnTaO₄:(Nb, Gd, Tm) series, Ln₂SiO₅ series, LnAlO₃:Ce series, LnOX:(Tb, Tm) series (X standing for a halogen), Ln₂O₃:Eu series, Ln₂O₂S:(Gd, Tb, Tm) series, CsX:Na series, CsX:Tl series, CsX:Eu series, BaFX:Eu series, ZnWO₄, HfP₂O₇ and Hf₃(PO₄)₃ can be employed. It is preferred that the phosphor be not lower than 7.0 in density, and especially preferably the phosphor is not lower than 9.0 in density. Examples of such a phosphor include LuTaO₄, LuTaO₄:Nb, Lu₂SiO₅, LuAlO₃:Ce, Lu₂O₂S:(Gd, Tb, Tm) series, Lu₂O₃: (Eu, Gd, Tb, Er, Tm) series, Gd₂O₃:(Tb, Tm) series, Gd₂O₂S:(Pr, Ce), CdWO₄, Gd₃Ga₅O₁₂:(Cr, Ce), HfO₂, TiCl:(Be, I) and Bi₄Ge₃O₁₂

As the material for the stimulable photoconductive layer 15, for instance, semiconductor materials disclosed in Japanese Unexamined Patent Publication No. 7(1955)-092023 can be employed and a polycrystalline film diamond can be preferably employed. As other examples, wide gap semiconductors such as single crystal diamond, SiC, BN (cubic), ZnO, ZnS, ZnSe, CdS, and AlN can be employed. These phosphors may be p-type, i-type or n-type and may be added with an impurity. Further, Si, Ge, Se, AlAs, AlSb, GaP, GaAs, GaSb, InP, InAs, PbO, CdSe, CdTe and other compound semiconductors can be employed and the laminates thereof can also be employed.

Further, materials known as the parent materials of the stimulable phosphors, e.g., BaFX (X=Cl, Br, I), CsX (X=Cl, Br, I), SrS, CaS, Zn₂SiO₄ and the like, can be used. These materials doped with an impurity to form a trap may be employed.

Further, other semiconductors, photo-chromic materials (“Optics”, an article by Tuyoshi Tsujioka, Vol. 32, No. 9, 2003, pp. 548 to 550) or photo-refractive materials may be employed. Specifically, inorganic materials such as GaN, BiI₃, Bi₁₂TiO₂₀, Bi₁₂GeO₂₀, Bi₁₂SiO₂₀, LiNbO₃, WO₃ and the like or organic materials such as Diarylethene (“Applied Physics Letters”, an article by Tuyoshi Tsujioka et al, Vol. 85, No. 15, 2004. Oct. 11, pp. 3128 to 3130) and the like may be employed.

As the EL layer 19, those which comprise (Ca•Sr)S:Eu²⁺ and contains phosphors. As the panel-like light source, for instance, that where an EL element disclosed in the pamphlet of International Patent Publication No. WO02/080626 is formed into a panel can be employed. Specifically, those having a light emitting layer where an electroluminescence light emitting particle group is dispersed in a dielectric binder as an EL layer. The “electroluminescence light emitting particle group” means a group of particles comprising phosphor particles, particles having a phosphor layer on the surface of dielectric particles, particles having a dielectric coating layer on the surface of phosphor particles, particles having a phosphor layer and a dielectric coating layer on the surface of dielectric particles, or combinations thereof. Light is emitted from the EL layer in response to application of an electric voltage across a pair of electrodes which are disposed on the respective sides of the EL layer. This emitted light is not only used as secondary stimulating light when the radiation image information is to be read out, but can be used as an erasing light when erasing the residual energy in the stimulable phosphor layer after reading out the image information. The second support 21 which is also a substrate of the panel-like light source may be formed of, for instance, PET and may be 10 to 1000 μm in thickness.

As the EL element employed in the panel-like light source, the following elements may be used in addition to those described above.

1) Elements which are disclosed in Japanese Unexamined Patent Publication No. 2004-311422 and comprise a substrate, a first electrode layer formed on the substrate, an insulating layer which is a thick film having a dielectric and is formed by injecting dielectric material onto the substrate on which the first electrode layer has been formed, an EL layer formed on the insulating layer and a second electrode layer formed on an upper layer of the EL layer.

2) Elements which are disclosed in Japanese Unexamined Patent Publication No. 2005-093358 and have a light emitting layer comprising an electroluminescence light emitting particle group (previously described) and filler which fills the spaces between the particles forming the particle group, and an insulating layer provided at least on one side of the light emitting layer between a pair of electrodes where the particles forming the particle group are in contact with each other or fused to each other, and the particle group is not smaller than 1.0 in the volume ratio to the filler in the light emitting layer.

3) Elements which are disclosed in Japanese Unexamined Patent Publication No. 2005-116503, have a light emitting layer which is not smaller than 15 μm in layer thickness and is dispersed with phosphor particles including luminescent center to be impacted and excited by hot electrons and start to emit light at an intensity of electric field not lower than 0.05 mV/cm.

4) Elements which are disclosed in Japanese Unexamined Patent Publication No. 2005-093359 and comprise a light emitting layer exhibiting field light emission and an insulating layer provided at least on one side of the light emitting layer, wherein the insulating layer is a layer which is not smaller than 0.5 μm and is not larger than 20 μm in thickness and includes first and second highly dielectric particle groups different in mean particle diameters at a packing of not smaller than 70% in total, the first highly dielectric particle groups being not smaller than 150 nm in the mean particle diameter and the second highly dielectric particle groups being not larger than a half of the first highly dielectric particle groups in the mean particle diameter.

5) Electroluminescence elements which are disclosed in Japanese Unexamined Patent Publication No. 2005-203336 and comprise a light emitting layer having a dielectric core and a phosphor coating layer which is provided outside the dielectric core and includes a number of electroluminescence light emitting particles where a mean intensity of field applied to the phosphor coating layer of the number of electroluminescence light emitting particles is not smaller than 1.5 times the mean intensity of field applied to the whole light emitting layer.

Though conventional dispersion type EL elements or the film type EL elements may be used as the EL element, the EL element can be brighter than the conventional EL elements by the use of the elements described above, a sharper image can be obtained by the use of a brighter EL element as a secondary stimulating light, and the erasing efficiency can be increased by the use of a panel-like light source having a brighter element as an erasing light source to be described later. As the EL element, organic EL elements may be used without limited to the inorganic EL elements.

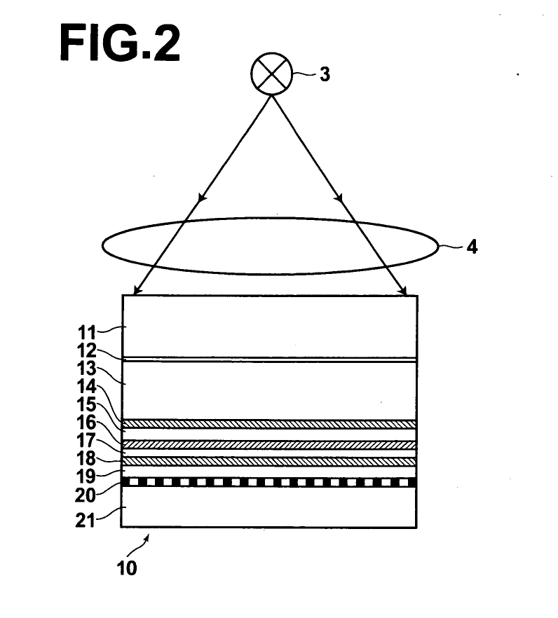

FIG. 2 is a schematic view for illustrating a radiography by the use of the radiation image information detecting panel 10 described above. Recording of radiation image information on the radiation image information detecting panel 10 will be described first.

Upon radiography, the radiation image information detecting panel 10 is positioned so that the first support 11 of the radiation image information detecting panel 10 is opposed to an object 4. The object 4 is exposed to radiation such as x-rays from a radiation source 3, and the radiation passing through the object 4, that is, radiation carrying thereon radiation image information of the object 4, passes through the first support 11 and the reflective layer 12 and impinges upon the radiation absorbing phosphor layer 13. The radiation absorbing phosphor layer 13 emits light according to the amount of radiation impinging thereupon, and the light impinges upon the stimulable photoconductive layer 15. The stimulable photoconductive layer 15 generates the electron-positive hole pairs in an amount according to the amount of light impinging thereupon and the electric charges thereof are trapped by the trap level. That is, the stimulable photoconductive layer 15 stores and records radiation image information by storing electric charges according the amount of energy of radiation. The radiation image information detecting panel 10 may be positioned so that the radiation such as x-rays impinges thereupon from the second support 21.

Upon taking a radiation image, since it is unnecessary to apply an electric field to the stimulable photoconductive layer or the like, the radiation image information detecting panel 10 may be handled in asynchronization with the radiograph system in the same manner as a conventional cassette which is loaded with a stimulable phosphor sheet and which is not provided with a detecting portion or a panel-like light source.

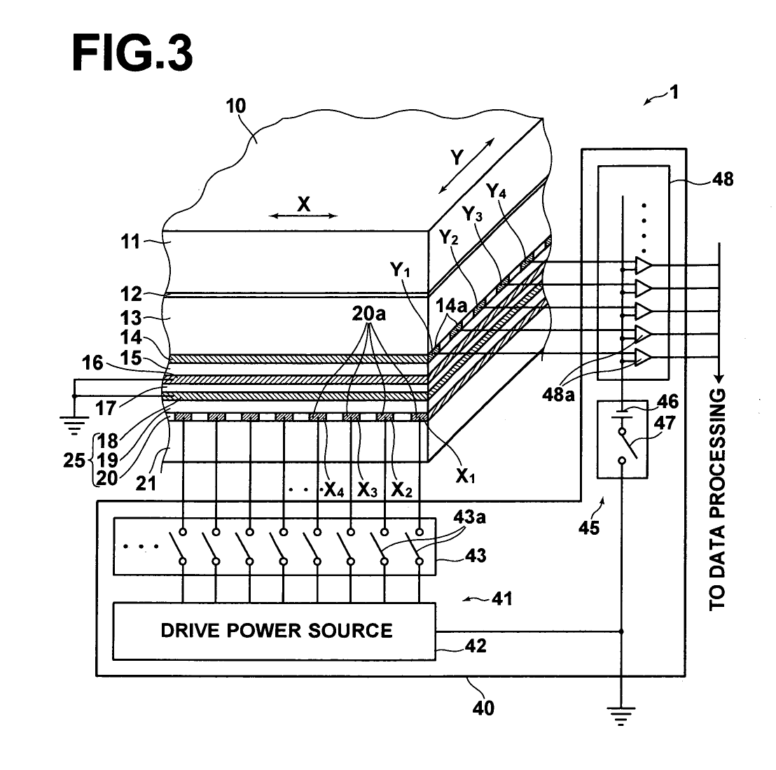

FIG. 3 is a view showing in brief a radiation image information read-out system 1 employing the radiation image information detecting panel 10.

The radiation image information read-out system 1 carries out scanning of the stimulating light by controlling the radiation image information detecting panel 10 and the panel-like light source 25 and t's provided with an image signal obtaining means 40 which obtains an image signal carrying thereon radiation image information by detecting electric charges generated in the stimulable photoconductive layer 15 in response to scanning of the stimulating light as a photocurrent by way of the detecting electrodes 14 and 16. The “photocurrent” as used here means an electric current generated by photo-stimulation.

The image signal obtaining means 40 is provided with a stimulating light source control means 41 for control of scanning of the stimulating light by the panel-like light source 25 and a current detecting means 45 which is provided with a current detecting circuit.

The stimulating light source control means 41 applies a predetermined electric field between the second flat plate electrode 18 and the second stripe electrode 20 for each element 20a of the second stripe electrode 20, or simultaneously for all or a plurality of elements 20a and comprises a drive power source portion 42 which outputs a drive voltage and a switching means 43 which switches the elements 20a which applies the drive voltage output from the drive power source portion 42 to the EL element 19. The second flat plate electrode 18 is grounded.

The switching means 43 is electrically connected to the drive power source portion 42 and is provided with a plurality of switching elements 43a, whereby each element is electrically turned on to the drive power source portion and off from the same in response to ON/OFF of the switching elements 43a.

When image is to be read out, the switching means 43 connects in sequence the elements 20a arranged in the scanning direction (the direction of arrow X) with the drive power source portion 42, that is, connects the drive power source portion 42 to the elements 20a in the order of X1, X2, X3 . . . . With this arrangement, the drive voltage is applied from the drive power source portion 42 to the elements 20a arranged in the direction of arrow X in sequence, and line light from the area between the element 20a and the flat plate electrode layer 18 is caused to scan in the direction of arrow X.

The current detecting means 45 comprises a voltage imparting means having a power source 46 for applying a voltage across the first stripe electrode layer 14 and the first flat plate electrode layer 16 in order to generate an electric field in the stimulable photoconductive layer 15 and a switch 47 and a current detecting amplifier portion 48 provided with a plurality of current detecting amplifiers 48a each connected to one of the elements 14a of the first stripe electrode layer 14.

The voltage imparting means closes the switch 47 (turning ON the switch 47) under the instruction of a control means (not shown) to apply a DC voltage across the electrode layers 14 and 16.

The current detecting amplifiers 48a detects in parallel as photocurrents electric charges generated in the stimulable photoconductive layer 15 in response to exposure to the stimulating light for each of the elements 14a. The first stripe electrode 14 is connected to the negative pole of the power source 46 by way of the connecting means 47 and the positive pole of the power source 46 is connected to each of the current detecting amplifiers 48a. The current detecting means 45 has a function of detecting photocurrents by electric charges generated in the stimulable photoconductive layer 15 in response to scanning of the stimulating light and obtaining an image signal carrying thereon radiation image information stored in the stimulable photoconductive layer 15.

Read-out of radiation image information stored in the radiation image information detecting panel 10 in the radiation image read-out system 1 will be described, hereinbelow.

When the radiation image information stored and recorded in the radiation image information detecting panel 10 is to be read out, the switch 47 is closed (turned ON) and in a state where a DC voltage is applied across the electrode layers 14 and 16, a predetermined voltage is applied across each of the elements 20a and the second the flat plate electrode 18 while the elements 20a is switched in sequence in the direction of arrow X by the light source control means 41. EL light is emitted from the EL layer 19 between the elements 20a and the second flat plate electrode 18 in response to application of the electric voltage. Since each element 20a is like a line extending from an end of the panel to the other end of the panel, the EL light is employed as a linear stimulating light. That is, the panel-like light source becomes equivalent to an electrode which is formed by two-dimensionally arranging the linear electrodes and by causing the electrode to switch in sequence the elements 20a, the stimulable photoconductive layer 15 is scanned in the X-direction (the main-scanning direction) with a linear stimulating light extending in the Y-direction (the sub-scanning direction.

The stimulating light impinges upon the stimulable photoconductive layer 15, and the electrons and the positive holes which have been trapped by trap levels are released in response to exposure to the stimulating light in the stimulable photoconductive layer 15. Since an electric field has been applied to the stimulable photoconductive layer 15, the electrons and the positive holes respectively move to the positive pole and the negative pole and are detected by the current detecting amplifier 48a as a photocurrent. At this time, the electric current is obtained as change in voltage observed at an output portion of the current detecting amplifier 48a.

Since the current detecting amplifier 48a is connected for each element 14a, a simultaneous read-out is carried out in the sub-scanning direction (the direction of arrow Y) in which the elements 14a are arranged. An image signal is obtained by detecting as a photocurrent the electric charges generated in sequence in the X-direction in response to the main scanning of the stimulating light, that is, the sequential switching of the elements 20a by the switching means 43. The image signal obtained by the image signal obtaining means 40 is input a data processing portion and a predetermined image processing is carried out on the image signal, whereby a visible image is displayed on a display means on the basis of radiation image information.

In the radiation image read-out system of this embodiment, since deterioration of S/N due to mingle of the stimulated light with the stimulating light does not take place, an image better in S/N than when the radiation image information is obtained by detecting the stimulated light in response to projection of the stimulating light can be obtained. Further, since the radiation image read-out system is provided with the panel-like light source, it is not necessary to provide an additional stimulating light projecting means, whereby the radiation image read-out system can be compactly arranged.

The electric charges are not sometimes wholly converted to the photocurrent and a part of the electric charges remain trapped in the trap levels of the stimulable photoconductive layer 15. When another image is taken as the electric charges remain in the trap levels, the latent image on the basis of the following radiation image information is added with the preceding latent image and a problem of an afterimage phenomenon or deterioration in S/N will arise.

Accordingly, before another image is taken after read-out of one image, the residual electric charges remaining trapped is released by projecting predetermined light (erasing light) onto the stimulable photoconductive layer 15 in the state where an electric field is applied to the stimulable photoconductive layer 15.

In the radiation image read-out system 1 of this embodiment, the panel-like light source for projecting the stimulating light may be used as the erasing light source for projecting the erasing light. Though the erasing light source may be separately provided, the number of parts of the system can be small and the system may be inexpensive if the erasing light and the stimulating light are emitted from one light source.

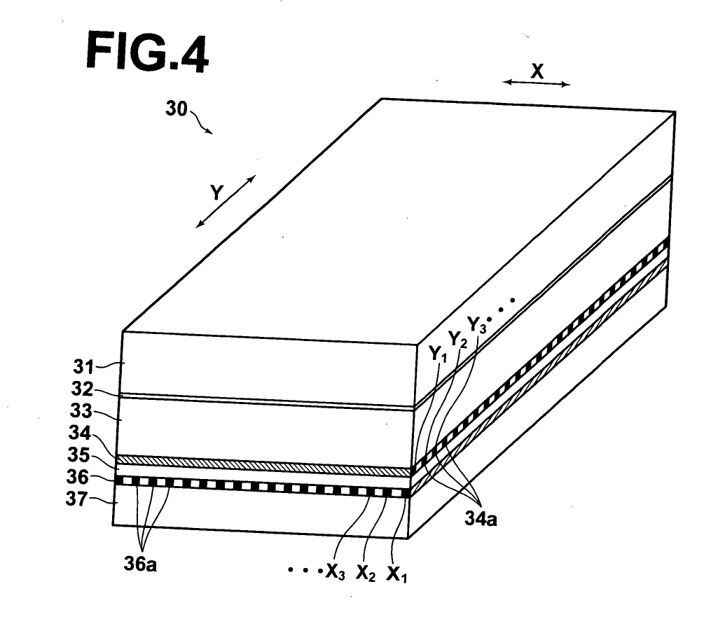

A radiation image information detecting panel in accordance with a second embodiment of the present invention will be described, hereinbelow. FIG. 4 is a perspective view of the radiation image information detecting panel 30 in accordance with the second embodiment of the present invention. The radiation image information detecting panel 30 comprises a first support (base) 31, a reflective layer 32, a radiation absorbing phosphor layer 33 which includes therein phosphors which absorb radiation and emit light according to the amount of the radiation, a first stripe electrode layer 34 which is formed by a number of linear electrodes (elements) 34a disposed like a stripe, a stimulable photoconductive layer 35 which absorbs light emitted from the radiation absorbing phosphor layer 33 to store energy according to the amount of the light, thereby storing and recording radiation image information, while generates electric charges according to the stored energy when an electric field is applied, a second stripe electrode layer 36 which is formed by a number of linear electrodes (elements) 36a disposed like a stripe, and a second support 37 which is a protective layer, and the layers are superposed and integrated in this order. Detail of each layer will be described later. In the panel 30 with this arrangement, it is preferred in obtaining a sharp image that the thickness of the stimulable photoconductive layer 35 is not larger than about 20 μm.

In the panel 30 of this embodiment, a pair of electric current detecting electrode layers for detecting the electric charges generated in the stimulable photoconductive layer 35 are formed by the first stripe electrode layer 34 and the second stripe electrode layer 36.

The elements 34a of the first stripe electrode layer 34 and the elements 36a of the second stripe electrode layer 36 cross with each other in perpendicular to the other as seen in plan. The elements 34a of the first stripe electrode layer 34 are linear electrode extending in the direction of arrow X from one end of the panel to the other at predetermined pitches in the direction of arrow Y (Y1, Y2, Y3, . . . ) and the elements 36a of the second stripe electrode layer 36 are linear electrode extending in the direction of arrow Y from one end of the panel to the other at predetermined pitches in the direction of arrow X (X1, X2, X3, . . . ). As in the first embodiment, the pitches of the elements 36a of the second stripe electrode layer 36 and the pitches of the elements 34a of the first stripe electrode layer 34 govern the pixel pitches in the main scanning direction (the direction of arrow X) and the sub-scanning direction (the direction of arrow Y) and the elements are arranged in same number as the desired number of the pixels.

The first stripe electrode layer 34 is permeable at least to light emitted from the radiation absorbing phosphor layer 31. These electrodes permeable to light may be formed by an electrode partially exhibiting permeability to light such as a mesh-like or stripe-like metal electrode or a combination of these electrodes as well as ITO (Indium Tin Oxide), indium-doped zinc oxide film or metal film.

The first support 31, the reflective layer 32 and the radiation absorbing phosphor layer 33 can be formed by the same material in the same thickness as the first support 11, the reflective layer 12 and the radiation absorbing phosphor layer 13 in the first embodiment. Further, the second support 37 may be formed by PET and has a thickness of, for instance, about 100 μm.

The stimulable photoconductive layer 35 may be formed, for instance, by materials ZnS:Cu, ZnS:Mn,Cu, ZnS:Mn,Pb,Cl, SrS:Eu,Sm. Ki:Cu and CdS:Ag disclosed in U.S. Pat. No. 7,087,915 which exhibits a Gudden-Pohl effect. These materials has a function of electric current flowing therethrough in response to Gudden-Pohl effect by application of an electric field. Since it is not necessary to have a light emitting function, impurities having a light emitting function may be replaced with impurities not having a light emitting function.

Taking a radiation image by the use of the radiation image information detecting panel 30 of the second embodiment can be carried out in the same procedure as taking a radiation image by the use of the radiation image information detecting panel 10 of the first embodiment.

Upon radiography, the radiation image information detecting panel 30 is positioned so that the first support 31 of the radiation image information detecting panel 30 is opposed to an object. The object is exposed to radiation such as x-rays from a radiation source, and the radiation passing through the object, that is, radiation carrying thereon radiation image information of the object, passes thorough the first support 31 and the reflective layer 32 and impinges upon the radiation absorbing phosphor layer 33. The radiation absorbing phosphor layer 33 emits light according to the amount of radiation impinging thereupon, and the light impinges upon the stimulable photoconductive layer 35. The stimulable photoconductive layer 35 stores and records radiation image information by storing energy according the amount of incident light.

Also in this case, since it is unnecessary to apply an electric field to the stimulable photoconductive layer or the like upon taking a radiation image, the radiation image information detecting panel 30 may be handled in asynchronization with the radiograph system in the same manner as a conventional cassette which is loaded with a stimulable phosphor sheet and which is not provided with a detecting portion or a panel-like light source.

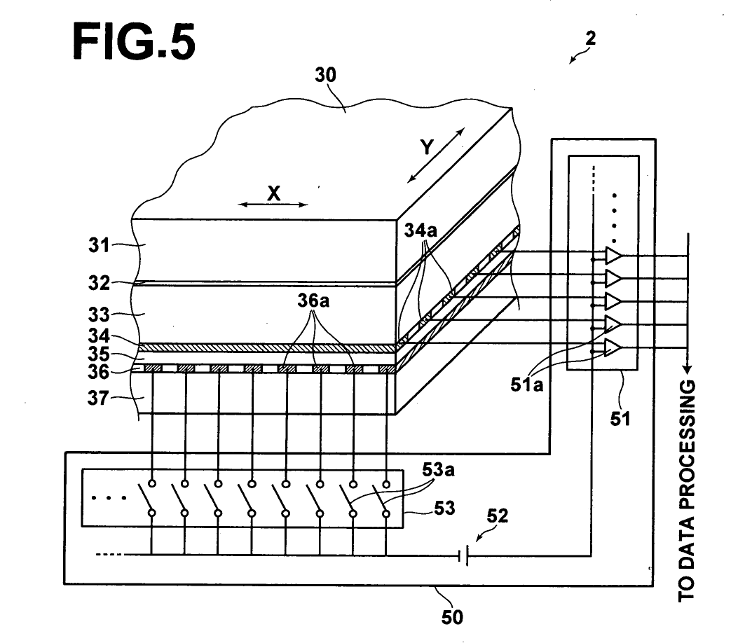

FIG. 5 is a view showing in brief a radiation image information read-out system 2 employing the radiation image information detecting panel 30.

The radiation image information read-out system 2 comprises the radiation image information detecting panel 2 shown in FIG. 4, and an image signal obtaining means 50 which controls application of an electric field to the stimulable photoconductive layer 35 and at the same time obtains an image signal carrying thereon radiation image information by detecting electric charges generated in the stimulable photoconductive layer 35 in response to application of the electric field by way of the electrode layers 34 and 36.

The image signal obtaining means 50 is provided with a current detecting amplifier portion 51 provided with a plurality of current detecting amplifiers 51a each, a power source 52, a switching means 53 which is provided with a plurality of switching elements 53a, each of which is connected to one of the elements 36a of the second stripe electrode layer 36 and a control means not shown.

The positive pole of the power source 52 is connected to each of the current detecting amplifiers 51a and the negative pole of the power source 52 is connected in common to one terminals of the switching elements 53a.

The power source 52 and the switching means 53 is for applying a predetermined electric voltage between the first and second stripe electrodes 34 and 36, and in the switching means 53, when radiation image information is to be read out, the elements 36a of the second stripe electrode 26 are connected to the respective elements 34a of the first stripe electrode 34 while the elements 36a of the second stripe electrode 26 are switched in sequence. The switching of the elements 53a of the switching means 53 is carried out by the control means not shown.

The current detecting amplifier portion 51 has a function of detecting electric charges generated in the stimulable photoconductive layer 35 in response to application of the electric voltage as an electric current generated by movement of the electric charges with each of the amplifiers 51a and obtaining an image signal carrying thereon radiation image information stored in the stimulable photoconductive layer 35 (distribution of the radiation energy two-dimensionally stored).

Read-out of radiation image information recorded in the radiation image information detecting panel 30 in the radiation image read-out system 2 will be described, hereinbelow.

When the radiation image information stored and recorded in the radiation image information detecting panel 30 is to be read out, the switching means 53 switches the elements 36a of the second stripe electrode 36 in sequence in the direction of arrow X from one end of the panel to the other end to apply an electric field to the linear area along the elements in sequence.

In the area of the stimulable photoconductive layer 35 applied with an electric field, electric charges are generated according to the energy stored in the area and the electric charges are detected by the current detecting amplifiers 51a by way of the electrode layer.

Since the current detecting amplifier 51a is connected for each element 34a, a simultaneous read-out is carried out in the sub-scanning direction (the direction of arrow Y) in which the elements 34a are arranged. An image signal on the basis of the radiation image information which has been stored and recorded as a two-dimensional energy distribution is obtained by detecting the electric charges generated in sequence in the X-direction in response to the sequential switching of the elements 36a by the switching means 53. The image signal obtained by the image signal obtaining means 50 is input a data processing portion and a predetermined image processing is carried out on the image signal, whereby a visible image is displayed on a display means on the basis of radiation image information.

In the radiation image read-out system 2 of this embodiment, since the optical stimulation is not carried out and the electric charges generated by the electric field stimulation are detected, it is not necessary to provide an additional stimulating light source, whereby the radiation image information detecting panel can be very simple in structure. Since deterioration of S/N due to mingle of the stimulated light with the stimulating light does not take place, an image better in S/N than when the radiation image information is obtained by detecting the stimulated light in response to projection of the stimulating light can be obtained.

The energy is not sometimes wholly released and a part of the energy remain in the stimulable photoconductive layer 35. When another image is taken as the energy remains in the stimulable photoconductive layer 35, the energy on the basis of the following radiation image information is added with the residual energy and a problem of an afterimage phenomenon or deterioration in S/N will arise. Accordingly, it is preferred that before another image is taken after read-out of one image, the residual energy be released by projecting predetermined light (erasing light) onto the stimulable photoconductive layer 35.

Further, it is preferred that either of the stimulable photoconductive layers 15 and 35 provided in the radiation image information detecting panels in accordance with the first and second embodiments described above be formed of a composite material including film or particles of the previously mentioned material and it is especially preferred that they be formed of an organic/inorganic composite material including inorganic particles in the organic material. It is further preferred that the particles have a particle diameter of nano order. The composite material is advantageous in that (1) a flexible layer can be formed by printing, application or ink jet (2) can be easily laminated with other functional layers and the device can be produced at low cost (3) there are larger amount of options from the viewpoint of practice since the inorganic semiconductors are larger in the mobility than organic semiconductors and there are stable semiconductors in inorganic semiconductors more than in organic semiconductors.

What is claimed is:

1. A radiation image information detecting panel comprising
a radiation absorbing phosphor layer including phosphors which absorb radiation and emit light according to the amount of the radiation,
a stimulable photoconductive layer which generates electron-positive hole charges in an amount according to the amount of light and stores and records a radiation image information by trapping the charges while releasing the trapped charges upon exposure to stimulating light,
a pair of photocurrent detecting electrode layers which are permeable to light and are respectively provided on upper and lower surfaces of the stimulable photoconductive layer to detect the released charges, and
a panel-like light source for projecting the stimulating light onto the stimulable photoconductive layer.

2. A radiation image information detecting panel as defined in claim 1 in which the panel-like light source comprises an electroluminescence layer emitting the stimulating light and a pair of electric field applying electrode layers at least the stimulable photoconductive layer side one of which is permeable to light and which are respectively provided on upper and lower surfaces of the electroluminescence layer to apply an electric field to the electroluminescence layer.

3. A radiation image information detecting panel as defined in claim 2 in which at least one of the photocurrent detecting electrode layers forms a first stripe electrode layer comprising a plurality of linear electrodes disposed parallel to each other and the other one of the photocurrent detecting electrode layers forms a second stripe electrode layer comprising a plurality of linear electrodes disposed parallel to each other, the linear electrodes of the first stripe electrode being in perpendicular to those of the second stripe electrode.

4. A radiation image information read-out system comprising a radiation image information detecting panel provided with a radiation absorbing phosphor layer including phosphors which absorb radiation and emit light according to the amount of the radiation, a stimulable photoconductive layer which generates electron-positive hole charges in an amount according to the amount of light and stores and records a radiation image information by trapping the charges while releasing the trapped charges upon exposure to stimulating light, a pair of photocurrent detecting electrode layers which are permeable to light and are respectively provided on upper and lower surfaces of the stimulable photoconductive layer to detect the released charges, and a panel-like light source for projecting the stimulating light onto the stimulable photoconductive layer, and
an image signal obtaining means which obtains an image signal carrying thereon image information by controlling the panel-like light source to cause the stimulating light to scan the stimulable photoconductive layer and detecting the charge released from the stimulable photoconductive layer in response to exposure to the stimulating light as a photocurrent by way of the photocurrent detecting electrode layers.

5. A radiation image information detecting panel comprising
a radiation absorbing phosphor layer including phosphors which absorb radiation and emit light according to the amount of the radiation,
a stimulable photoconductive layer which generates electron-positive hole charges in an amount according to the amount of light and stores and records a radiation image information by trapping the charges while releasing the trapped charges upon application of an electric field, and
a pair of photocurrent detecting electrode layers which are respectively provided on upper and lower surfaces of the stimulable photoconductive layer to apply the electric field and detect the charges, at least the stimulable photoconductive layer side one of the photocurrent detecting electrode layers being permeable to light.

6. A radiation image information detecting panel as defined in claim 1 in which the pair of photocurrent detecting electrode layers is of stripe electrodes each of which comprises a plurality of linear electrodes parallel to each other, and disposed so that the linear electrodes thereof extends perpendicularly to each other.

7. A radiation image information read-out system comprising a radiation image information detecting panel provided with a radiation absorbing phosphor layer including phosphors which absorb radiation and emit light according to the amount of the radiation, a stimulable photoconductive layer which generates electron-positive hole charges in an amount according to the amount of light and stores and records a radiation image information by trapping the charges while releasing the trapped charges upon application of an electric field, and a pair of photocurrent detecting electrode layers which are respectively provided on upper and lower surfaces of the stimulable photoconductive layer to apply the electric field and detect the charges, at least the stimulable photoconductive layer side one of the photocurrent detecting electrode layers being permeable to light, and
an image signal obtaining means which obtains an image signal carrying thereon image information by controlling the electric field applied to the stimulable photoconductive layer and detecting the charge released from the stimulable photoconductive layer in response to application of the electric field by way of the photocurrent detecting electrode layers.
